(19) United States
(12) Patent Application Publication
(10) Pub. No.: US 2016/0256052 A1
(43) Pub. Date: Sep. 8, 2016

(54) INTRAOCULAR PRESSURE DETECTING DEVICE

(71) Applicant: CRYSTALVUE MEDICAL CORPORATION, Taoyuan City (TW)

(72) Inventors: Wen Wei HUANG, Taoyuan City (TW); Chung Ping CHUANG, Taoyuan City (TW)

(73) Assignee: CRYSTALVUE MEDICAL CORPORATION, Taoyuan City (TW)

(21) Appl. No.: 14/637,073

(22) Filed: Mar. 3, 2015

(51) Int. Cl. A61B 3/16; A61B 3/18; A61B 3/10

(52) U.S. Cl. CPC A61B 3/165; A61B 3/1005; A61B 3/18

(57) ABSTRACT

The present invention provides an intraocular pressure detecting device, which includes: a sampling device, a comparison device and a detecting device. The sampling device includes an opening window, an imaging unit and a puffing unit. The opening window has a through hole. The imaging unit forms an imaging optical path directly with the eyeball, via a through hole of the opening window. In addition, the puffing device forms a puffing path together with the through hole of the opening window towards the eyeball. Moreover, the comparison device includes a reflecting mirror and a driving device, and the driving device drives the reflecting mirror to generate a displacement. The detecting device is in connection with the sampling device and the comparison device. The detecting device forms a detecting optical path with the eyeball by means of a through hole of the opening window. Also, the detecting device forms a comparison optical path with the reflection mirror of the comparison device. Further, the detecting device projects a first detecting signal that is faced towards the detecting optical path and a second detecting signal that is faced towards the comparison optical path. The first detecting signal forms a first reflecting signal, and the second detecting signal forms a second reflecting signal. As such, a value of an intraocular pressure and a thickness of a cornea of the test subject are calculated by the detecting device that receives the first reflecting signal and the second reflecting signal.

### FIELD OF THE INVENTION

The present invention relates generally to a detection device that is able to carry out constant-pressure puffing towards the eye of a test subject, and more particularly, the present invention relates to a detection device that is able to calculate the value of intraocular pressure and the thickness of cornea of the eyeball of a test subject at the same time.

### BACKGROUND OF THE INVENTION

Many tools exist for the detection of intraocular pressure of the eyeball of test subjects, but the common tools that are normally used for detecting intraocular pressure include an applanation tonometer, a tonopen as well as a pneumatonometer. The most reliable method for the detection of intraocular pressure is the applanation tonometer. However, to detect the intraocular pressure of the eyeball of the test subject with a applanation tonometer, anesthetic needs to be applied on the cornea of the eyeball of the test subject before the detection method, and the intraocular pressure of the test subject may be detected by subsequently placing the applanation tonometer in direct contact with the cornea of the eyeball of a test subject.

As to the tonopen, the tonopen has a design that is similar to the design of an applanation tonometer. In other words, to detect the intraocular pressure of the eye of a test subject, the tonopen needs to be in direct contact with the eye. The main advantages of the tonopen are that it is easily portable, and that the speed of detecting the intraocular pressure is fast. However, the rate of failure and the error rate are relatively high.

With regard to the detection method by the pneumatonometer, this detection method involves the instantaneous injection of a air with a certain pressure to the cornea of the eyeball of the test subject in order to flatten the cornea of the eyeball of the test subject; this detection method makes use of electrons to detect the changes in reflected wave, and thus calculating the value of the intraocular pressure. The main advantage of the pneumatonometer is that no direct contact with the test subject's cornea is needed, however, errors would occur with this detection method when the intraocular pressure gets too high, for example at thirty to forty millimeters of mercury. As such, the pneumatonometer is used mainly for screening.

Referring to FIG. 1, the conventional pneumatic tonometers have a slit plate 11 in front of the eyeball 10, and a first lens 12 and a second lens 13 exist sequentially behind the slit plate 11. An imaging optical path 15 is formed directly by a light-sensitive element 14 which is behind the second lens 13, whereby the puffing device (not shown in the Figs.) is set up in between the slit plate 11 and the first lens 12, and air is passed through the air space of the slit plate 11 in order for a puffing path 16 to be formed, enabling air to be puffed directly towards the eyeball 10.

Moreover, the detecting optical path 17 of the conventional pneumatic tonometer may project an infrared light source 18 to the eyeball 10 in a different direction to the puffing device, such that the value of an intraocular pressure can be calculated by the photoelectric cell 19 that receives a signal reflected by the eyeball 10.

However, since the detecting optical path and the puffing path of the conventional pneumatic tonometer are located in different paths, the errors which are caused by the tolerances of the components and the assembly errors may result from the errors of the result determined. As such, in order to enable the conventional pneumatic tonometer to calculate a value of an intraocular pressure more accurate, it is necessary to have an improvement on the detecting optical path and the imaging optical path of the conventional determined tonometer.

### SUMMARY OF THE INVENTION

The main objective of the present invention is to enable the values of intraocular pressure and thickness of the cornea of the eyeball of the test subject to be detected at the same time, by means of placing the detecting optical path and the comparison optical path within different device structures.

The other objective of the present invention is to have a design such that the detecting optical path and the puffing path may be on the same axis, so as to effectively reduce the errors that may be caused by the tolerance of the components.

In order to achieve the aforesaid objectives, the present invention is related to an intraocular pressure detecting device, for the detection of the intraocular pressure as well as the thickness of the cornea of the eyeball of test subjects, whereby the intraocular pressure detecting device of the present invention includes a sampling device, a comparison device, a detecting device as well as a gazing unit.

The sampling device may include an opening window that forms a through hole, whereby the interior of the sampling device may include an imaging unit as well as a puffing unit. The imaging unit may form an imaging optical path directly with the eyeball, via a through hole of the opening window.

According to a preferred exemplary embodiment of the present invention, the comparison device may include a reflecting mirror and a driving device; and the detecting device may be in connection with the sampling device and the comparison device, respectively. The detecting device may indirectly form a detecting optical path with the eyeball, via the opening window of the sampling device. In addition, in accordance with a preferred exemplary embodiment of the present invention, the detecting device may directly form a comparison optical path with the reflecting mirror of the comparison device. The comparison optical path may include a second relay lens, and the driving device may drive the reflecting mirror to selectively get close to or to get further away from the second relay lens.

Whereby, the detecting device may include a projecting element, a light splitting element as well as an operating element. The projecting element may include a first detecting signal and a second detecting signal. In addition, the light splitting element is in connection with the projecting element, whereby the light splitting element projects the first detecting signal to the detecting optical path, and projects the second detecting signal to the comparison optical path. Moreover, the light splitting element may receive the first reflecting signal and the second reflecting signal. Furthermore, the operating element is in connection with the light splitting element, the operating element may receive the first reflecting signal and the second reflecting signal, so as to calculate a current value for the intraocular pressure and value for the thickness of the cornea of the eyeball of the test subject.

In addition to the above, the detecting optical path and the imaging optical path may enable the imaging unit of the imaging optical path and the detecting device of the detecting optical path to be located on a different axis, by means of a first light splitting lens. Moreover, in accordance with a preferred exemplary embodiment of the present invention, a first relay lens may exist in between the first light splitting lens and the opening window. The puffing device may work together with the through holes of the opening window to blow air towards the eyeball of the test subject; furthermore, the puffing device may blow air towards the area between the opening window and the first relay lens, so as to form a puffing path that is on the same axis as the detecting optical path.

The gazing unit is located in the interior of the sampling device. Moreover, the gazing unit may form a gazing optical path with the eyeball of the test subject by means of the opening window. In addition, the gazing optical path and the detecting optical path may enable the gazing optical path and the detecting optical path to be located on a different axis, by means of a second light splitting lens.

In accordance with a preferred exemplary embodiment of the present invention, when the length of the comparison optical path is subjected to adjustment, such that this length is now equal to the length of the detecting optical path, the detecting device may calculate the value of the intraocular pressure by means of the first reflecting signal and the second reflecting signal.

The distinguishing technical feature of the present invention lies in the fact that the detecting optical path as well as the puffing path of the puffing device may be located on the same axis at the same time. This may enable the errors that may be caused by the tolerance of the components to be effectively reduced. In addition to the above, the detecting device may be able to calculate the values of intraocular pressure and the thickness of the cornea of the eyeball of the test subject at the same time, by means of receiving the first reflecting signal and the second reflecting signal.

### BRIEF DESCRIPTION OF THE DRAWINGS

The present invention can be understood in more detail by reading the subsequent detailed description in conjunction with the examples and preferred exemplary embodiments made to the accompanying drawings, wherein:

FIG. 1 is a schematic diagram showing an optical path of a conventional intraocular pressure detecting device for imaging purposes.

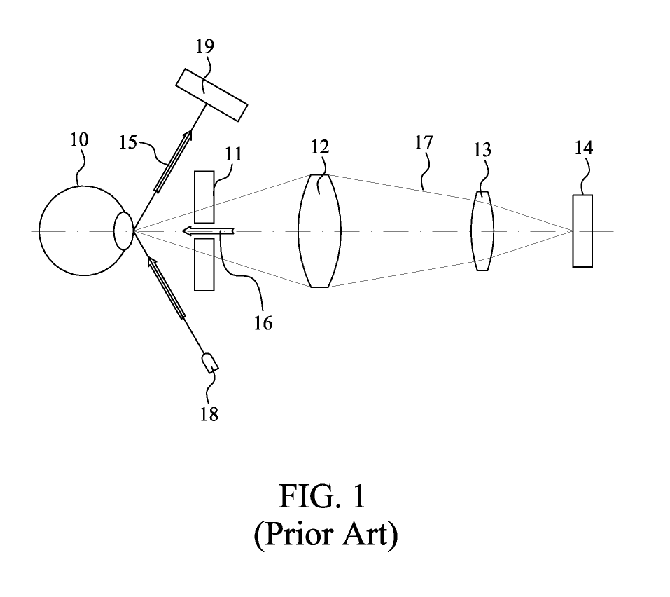

FIG. 2 is a schematic diagram showing the optical path of an intraocular pressure detecting device in accordance with a preferred exemplary embodiment of the present invention.

### DETAILED DESCRIPTION OF THE PREFERRED EMBODIMENT

The accompanying drawings are included to provide a further understanding of the invention, and are incorporated in and constitute a part of this specification. The drawings illustrate the preferred exemplary embodiments of the invention and, together with the description, serve to explain the principles of the invention.

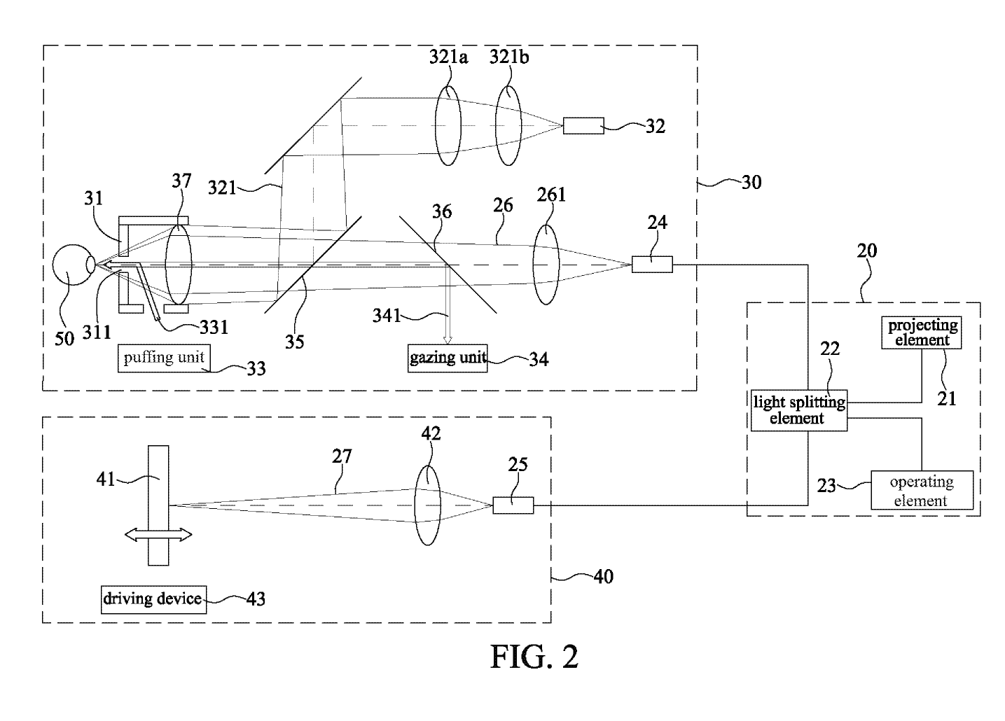

As shown in FIG. 2, in accordance with a preferred embodiment of the present invention, an intraocular pressure detecting device of the present invention, for the detection of the pressure of an eyeball and the thickness of a cornea of a eyeball of a test subject, is mainly composed of a detecting device 20, a sampling device 30 and a comparison device 40.

The detecting device 20 may include a projecting element 21, a light splitting element 22, an operating element 23, a first projecting element 24 and a second projecting element 25. The projecting element 21 may be in connection with the light splitting element 22. In addition, the projecting element 21 may include a first detecting signal and a second detecting signal. Moreover, the light splitting element 22 may be in connection with the operating element 23. The first projecting element 24 and the second projecting element 25 may respectively be in connection with the light splitting element 22. The first projecting element 24 may be located inside the sampling device 30, and the second projecting element 25 may be located inside the comparison device 40.

Also, as shown in FIG. 2, the sampling device 30 may be in connection with the detecting device 20. In addition to this, the sampling device 30 may include an opening window 31, an imaging unit 32, a puffing unit 33, a gazing unit 34, a first light splitting lens 35 and a second light splitting lens 36. Moreover, the opening window 31 may include a through hole 311. The imaging unit 32 may form an imaging optical path 321 directly with the eyeball of a test subject, by means of the through hole 311 of the opening window 31 and the first light splitting lens 35. A first relay lens 37 may be located in between the first light splitting lens 35 and the opening window 31. The puffing unit 33 may be located in between the opening window 31 and the first relay lens 37, and may form a puffing path 331 by means of puffing towards the eyeball of the test subject based on the through hole 311 of the opening window 31. Also, in accordance with the preferred exemplary embodiment of the present invention, the puffing unit 33 may form a puffing path 331 together with the through hole 311 of the opening window 31, enabling air to be blown towards the eyeball of the test subject.

In accordance with the preferred exemplary embodiment of the present invention, the operating element 23 of the detecting device 20 may be a charge coupled device (CCD). Moreover, the imaging unit 32 of the sampling device 30 may be a complementary metal oxide semiconductor (CMOS) image sensor.

Referring to FIG. 2, the gazing unit 34 may form a gazing optical path 341 with the eyeball 50 of the test subject by means of the second light splitting lens 36 and the through hole 311 of the opening window 31. The comparison device 40 may be in connection with the detecting device 20. In addition, the comparison device 40 may include a reflecting mirror 41, a second relay lens 42 and a driving device 43. The driving device 43 may drive the reflecting mirror 41 to selectively get close to or get further away from the second relay lens 42.

Moreover, the detecting device 20 may form a detecting optical path 26 directly with the eyeball 50 of the test subject, by means of the light splitting element 22, the first projecting element 24, the first light splitting lens 35, the first relay lens 37 and the through hole 311 of the opening window 31. It should be noted that the detecting optical path 26 and the puffing path 331 are located on the same axis. In addition, the detecting device 20 may form a comparison optical path 27, by means of the light splitting element 22, the second projecting element 25, the second relay lens 42 and the reflecting mirror 41.

In addition, as shown in FIG. 2, the imaging optical path 321 and the detecting optical path 26 may enable the imaging unit 32 of the imaging optical path 321 and the detecting device 20 of the detecting optical path 26 to be located on two different axises by means of the first light splitting lens 35. In addition, the gazing optical path 341 and the detecting optical path 26 may enable the gazing unit 34 and the detecting device 20 to be located on two different axises by means of the second light splitting lens 36.

In accordance with the preferred exemplary embodiment of the present invention, the imaging optical path 321 may include a third relay lens 321a and a fourth relay lens 321b. In addition, the third relay lens 321a and the fourth relay lens 321b may be located in between the imaging unit 32 and the first light splitting lens 35. Moreover, the detecting optical path 26 may include a fifth relay lens 261 that is located in between the first light splitting lens 35 and the first projecting element 24.

In the specific application, as shown in FIG. 2, the eyeball 50 of the test subject may be close to the through hole 311 of the opening window 31, and may gaze at the gazing unit 34 by means of the gazing optical path 341. In addition, the projecting element 21 of the detecting device 20 may continuously project the first detecting signal and the second detecting signal towards the light splitting element 22. At the same time, the light splitting element 22 may project the first detecting signal and the second detecting signal towards the first projecting element 24 and the second projecting element 25, respectively.

As shown in FIG. 2, the first projecting element 24 may project the first detecting signal towards the detecting optical path 26, and may form a first reflecting signal by means of a reflection from the eyeball 50 of the test subject. Subsequent to this, the first reflecting signal may be projected towards the light splitting element 22 of the detecting device 20 along the detecting optical path 26. Moreover, the driving device 43 may drive the reflecting mirror 41, so as to change the relative distance between the reflecting mirror 41 and the second relay lens 42, such that the lengths of the detecting optical path 26 and the comparison optical path 27 may be the same.

As such, in accordance with the preferred exemplary embodiment of the present invention, the second projecting element 25 may project the second detecting signal towards the comparison optical path 27, and may form a second reflecting signal corresponding to the first reflecting signal by means of reflection from the reflecting mirror 41 of the comparison device 40. Following this, the second reflecting signal may be projected towards the light splitting element 22 of the detecting device 20 along the comparison optical path 27. The light splitting element 22 of the detecting device 20 may also transmit the first reflecting signal and the second reflecting signal to the operating element 23, such that a value of an intraocular pressure and a thickness of a cornea of the eyeball 50 of the test subject may be calculated by the operating element 23 based on the first reflecting signal and the second reflecting signal.

This is then followed by the puffing of the puffing unit 33, which may puff based on the puffing path 331 towards the eyeball 50 of the test subject, so as to enable the eyeball 50 of the test subject to be inwardly compressed by air, such that the length of the detection optical path 26 may be increased. As such, when the first reflecting signal may not correspond to the second reflecting signal, the driving device 43 of the comparison device 40 may drive the reflecting mirror 41 to get further away from the second relay lens 42. In accordance with this, the length of the comparison optical path 27 may be equal to the length of the detecting optical path 26, so as to enable the first reflecting signal to correspond to the second reflecting signal. Therefore, the value of an intraocular pressure and a thickness of a cornea of the eyeball of the test subject may be calculated by the operating element 23 of the detecting device 20 by means of the first reflecting signal and the second reflecting signal.

In summary, the present invention may effectively reduce the error that is caused by the impact of part tolerances by the detecting optical path and the puffing path of the puffing device being located on the same axis. Furthermore, the value of intraocular pressure and the value of the thickness of a cornea of the eyeball of the test subject may be calculated by the detecting device that receives the first reflecting signal and the second reflecting signal.

Although the preferred exemplary embodiments of the present invention have been described with reference to the preferred exemplary embodiments thereof, it may be apparent to those ordinarily skilled in the art that a variety of modifications and changes may be made without departing from the scope of the present invention which is intended to be defined by the appended claims.

1. An intraocular pressure detecting device, for detecting an intraocular pressure of an eyeball and a thickness of a cornea of the eyeball of a test subject, comprising:
a sampling device having a through hole that forms an opening window, an interior of the sampling device including
an imaging unit that forms an imaging optical path extending toward the eyeball through the opening window, and
a puffing unit that forms a puffing path through the opening window, enabling air to be puffed towards the eyeball of the test subject;
a comparison device including
a reflecting mirror and
a driving device that drives a displacement of the reflecting mirror; and
a detecting device that communicates with the sampling device and the comparison device, respectively, the detecting device including:
a projecting element which generates a first detecting signal and a second detecting signal;
a light splitting element which
transmits the first detecting signal received from the projecting element to the sampling device to indirectly form a detecting optical path in the sampling device,
transmits the second detecting signal received from the projecting element to the comparison device to directly form a comparison optical path in the comparison device, and
receives a first reflecting signal that is formed from the first detecting signal reflected at the eyeball and a second reflecting signal that is formed from the second detecting signal reflected at the reflecting mirror, and
an operating element which
communicates with the light splitting element,
receives the first reflecting signal and the second reflecting signal, and
calculates the intraocular pressure and the thickness of the cornea of the eyeball of the test subject from the first and second reflecting signals.

2. (canceled)

3. The intraocular pressure detecting device in accordance to claim 1, wherein the puffing path and the detecting optical path are located on a position of the same axis.

4. The intraocular pressure detecting device in accordance to claim 1, further comprising a first light splitting lens which changes a first direction of a signal passing through the imaging optical path to a second direction of a signal passing through the detecting optical path.

5. The intraocular pressure detecting device in accordance to claim 4, further comprising a first relay lens which is arranged between the first light splitting lens and the opening window,
wherein the puffing unit puffs air towards the eyeball through the puffing path.

6. The intraocular pressure detecting device in accordance to claim 1, wherein a length of the comparison optical path is equal to the length of the detecting optical path.

7. The intraocular pressure detecting device in accordance to claim 1, wherein
the sampling device further comprises a gazing unit, the sampling device including a gazing optical path that is a path between the gazing unit and the opening window.

8. The intraocular pressure detecting device in accordance to claim 7, further comprising a second light splitting lens which changes a third direction of a signal passing through the gazing optical path to a fourth direction of a signal passing through the detecting optical path.

9. The intraocular pressure detecting device in accordance to claim 1, wherein the comparison device further comprises a second relay lens, and the driving device drives the reflecting mirror to selectively get close to or to get further away from the second relay lens.

10. The intraocular pressure detecting device in accordance to claim 1, wherein the puffing unit calculates a value of an intraocular pressure and a thickness of a cornea of the eyeball of the test subject.
